(19) United States
(12) Patent Application Publication
(10) Pub. No.: US 2026/0130962 A1
(43) Pub. Date: May 14, 2026

(54) TRADITIONAL CHINESE MEDICINE COMPOSITION AND TRADITIONAL CHINESE MEDICINE DECOCTION FOR PREVENTING AND/OR TREATING INTERSTITIAL LUNG DISEASE, AND PREPARATION METHOD THEREFOR AND USE THEREOF

(71) Applicants: Chongqing Academy of Chinese Materia Medica, Chongqing City (CN); First Affiliated Hospital of Army Medical University, Chongqing City (CN)

(72) Inventors: Na LI, Chongqing City (CN); Qinghua ZOU, Chongqing City (CN); Xingjian WEN, Chongqing City (CN); Xuefeng HE, Chongqing City (CN); Jie ZHU, Chongqing City (CN); Rui LIANG, Chongqing City (CN); Jiuyu FAN, Chongqing City (CN); Qi REN, Chongqing City (CN); Kang XIAO, Chongqing City (CN); Chengshun CHEN, Chongqing City (CN)

(73) Assignees: Chongqing Academy of Chinese Materia Medica, Chongqing City (CN); First Affiliated Hospital of Army Medical University, Chongqing City (CN)

(21) Appl. No.: 19/390,157

(22) Filed: Nov. 14, 2025

(30) Foreign Application Priority Data
Nov. 14, 2024 (CN) 202411624664.5

(51) Int. Cl. A61K 36/481; A61K 36/232; A61K 36/236; A61K 36/284; A61K 36/344; A61K 36/346; A61K 36/484; A61K 36/537; A61K 36/73; A61K 36/79; A61K 47/14

(52) U.S. Cl. CPC A61K 36/481; A61K 36/232; A61K 36/236; A61K 36/284; A61K 36/344; A61K 36/346; A61K 36/484; A61K 36/537; A61K 36/73; A61K 36/79; A61K 47/14; A61K 2236/331; A61K 2236/51

(57) ABSTRACT

A traditional Chinese medicine composition and a traditional Chinese medicine decoction are used for preventing and/or treating interstitial lung disease. The traditional Chinese medicine composition includes: 25-35 parts of Astragali Radix, 15-25 parts of Codonopsis Radix, 10-15 parts of Atractylodis Macrocephalae Rhizoma, 10-20 parts of Poria, 10-15 parts of Armeniacae Semen Amarum, 10-15 parts of Platycodonis Radix, 10-20 parts of Salviae Miltiorrhizae Radix et Rhizoma, 10-20 parts of Chuanxiong Rhizoma, 10-15 parts of Angelicae Sinensis Radix, 4-10 parts of Schisandrae Chinensis Fructus, and 5-12 parts of Glycyrrhizae Radix et Rhizoma. These ingredients together have the effects of strengthening and replenishing (the spleen and the lung), activating blood and dissolving stasis, and relieving cough and asthma, thereby preventing and/or treating interstitial lung disease.

### CROSS-REFERENCE TO RELATED APPLICATION

This patent application claims the benefit and priority of Chinese Patent Application No. 202411624664.5 filed with the China National Intellectual Property Administration on Nov. 14, 2024, the disclosure of which is incorporated by reference herein in its entirety as part of the present application.

### SEQUENCE LISTING

A computer readable XML file entitled “SEQUENCELISTING”, which was created on Nov. 12, 2025 and has a file size of about 3,206 bytes, contains the sequence listing for this application and is hereby incorporated by reference in its entirety.

### TECHNICAL FIELD

The present disclosure belongs to the technical field of traditional Chinese medicine, and in particular relates to a traditional Chinese medicine composition and a traditional Chinese medicine decoction for preventing and/or treating interstitial lung disease, and preparation methods therefor and the use thereof.

### BACKGROUND

Interstitial lung disease (ILD) is an umbrella term for a heterogeneous group of non-neoplastic, non-infectious lung diseases characterized by inflammation of alveoli, the basic respiratory units, and interstitial fibrosis. It is also known as diffuse parenchymal lung disease (DPLD). It often manifests as decreased respiratory function in patients, with clinical symptoms such as dyspnea, long-term cough and wheezing, and shortness of breath.

The high morbidity and mortality associated with ILD have become increasingly significant, yet effective therapeutic interventions remain limited. In Western medicine, the primary clinical interventions are oxygen therapy and anti-fibrosis treatments. Pirfenidone and nintedanib can delay the deterioration of lung function in patients with pulmonary fibrosis. However, in actual clinical use, the efficacy of the above drugs in treating idiopathic pulmonary fibrosis is slow to take effect and adverse effects remain challenging to manage. In addition, some new treatment methods have been developed, such as bone marrow stem cell transplantation, which can promote the repair of lung tissue structure and function. However, stem cell transplantation therapy is still in the experimental research stage and has a long way to go before it can be used in clinical practice.

In the theoretical system of traditional Chinese medicine, there is no specific disease that corresponds to ILD. Most people believe that ILD belongs to the category of “lung impediment”, with the disease locating in the lungs and spleen. Its main pathogenesis is the deficiency of the root and the excess of the superficial symptoms based on Qi deficiency in the lungs and spleen, and the method of reinforcing earth to generate metal is the main treatment principle and method. Huangdi Neijing (Inner Canon of Yellow Emperor) says: “the three Qi of wind, cold and dampness appear mingled. They bind to each other and form impediment”, “if skin impediment lingers and the patient is reinvaded by pathogenic factors, it attacks the lungs”, and “lung impediment manifests as restlessness, irritability, and fullness of the chest. This is coupled with dyspnea and vomiting”. This is the earliest record of the etiology and pathogenesis of lung impediment. Later generations of doctors also followed the theory of “lung impediment” in the Huangdi Neijing (Inner Canon of Yellow Emperor), and from the relevant discussion, it can be seen that internal deficiency of the viscera is the key to the occurrence of lung impediment. Currently, reports on the effectiveness of traditional Chinese medicine in preventing and treating ILD mainly focus on clinical efficacy and preclinical research into related mechanisms, including reducing oxidative stress, improving inflammation, inhibiting epithelial-mesenchymal transition and myofibroblast activation, and regulating autophagy and apoptosis.

### SUMMARY

In view of this, the object of the present disclosure is to provide a traditional Chinese medicine composition and a traditional Chinese medicine decoction for preventing and/or treating interstitial lung disease, and preparation methods therefor and the use thereof. The traditional Chinese medicine composition or traditional Chinese medicine decoction can improve lung function, inhibit pulmonary fibrosis and inflammation, and modulate the intestinal flora, thereby preventing and/or treating interstitial lung disease.

To achieve the above object of the present disclosure, the present disclosure provides the following technical solutions:

The present disclosure provides a traditional Chinese medicine composition for preventing and/or treating interstitial lung disease, including the following raw materials in parts by weight:

25-35 parts of Astragali Radix, 15-25 parts of Codonopsis Radix, 10-15 parts of Atractylodis Macrocephalae Rhizoma, 10-20 parts of Poria, 10-15 parts of Armeniacae Semen Amarum, 10-15 parts of Platycodonis Radix, 10-20 parts of Salviae Miltiorrhizae Radix et Rhizoma, 10-20 parts of Chuanxiong Rhizoma, 10-15 parts of Angelicae Sinensis Radix, 4-10 parts of Schisandrae Chinensis Fructus, and 5-12 parts of Glycyrrhizae Radix et Rhizoma.

In a preferred embodiment, the traditional Chinese medicine composition includes the following raw materials in parts by weight:

28-32 parts of Astragali Radix, 16-23 parts of Codonopsis Radix, 11-14 parts of Atractylodis Macrocephalae Rhizoma, 12-17 parts of Poria, 11-13 parts of Armeniacae Semen Amarum, 11-13 parts of Platycodonis Radix, 12-18 parts of Salviae Miltiorrhizae Radix et Rhizoma, 13-17 parts of Chuanxiong Rhizoma, 11-14 parts ofAngelicae Sinensis Radix, 5-7 parts of Schisandrae Chinensis Fructus, and 8-10 parts of Glycyrrhizae Radix et Rhizoma.

The traditional Chinese medicine composition for treating ILD in the present disclosure takes “Qi, deficiency and blood stasis” as the core pathogenesis of ILD in the prescription, with the primary treatment task being to address Qi deficiency and blood stasis. In other words, the focus is on addressing the underlying deficiency of the root and the excess of the superficial symptoms. The root cause lies in the lungs and spleen. Visceral weakness leads to the obstruction of the meridians, resulting in the retention of pathological products such as blood stasis and phlegm, which further damage the healthy Qi. Ultimately, the accumulated damage becomes irreparable and the lungs lose their function. Therefore, the treatment method should take into account both strengthening healthy Qi (strengthening the spleen and tonifying the lungs) and eliminating pathogens (activating blood and dissolving stasis and eliminating phlegm).

The prescription interpretation of the traditional Chinese medicine composition for preventing and/or treating interstitial lung disease in the present disclosure is as follows:
With regard to the monarch medicine: Astragali Radix was first recorded in the Divine Farmer's Materia Medica. It tastes sweet and is slightly warm in nature. It enters the spleen and lung meridians and is often used to strengthen healthy Qi and consolidate body resistance. Astragali Radix contains many effective active ingredients, among which total saponins, including astragaloside IV, astragalus polysaccharides, astragalus glycoprotein, astragalus flavone, exert different degrees of effects on regulating immunity, combating inflammation, and inhibiting epithelial-mesenchymal transition. Astragali Radix is widely used in the treatment of pulmonary fibrosis. The use of Codonopsis Radix as herbal medicine was first recorded in the New Compendium of Materia Medica of the Qing Dynasty. Codonopsis Radix tastes sweet and is warm in nature; it enters the spleen and lung meridians, and has the efficacy of strengthening the spleen and tonifying the lungs, and nourishing blood and producing body fluid. It can inhibit high epithelial-mesenchymal transition and inflammatory reaction. Astragali Radix, Codonopsis Radix, Atractylodis Macrocephalae Rhizoma and Poria are used in combination as the monarch medicine and have the effects of invigorating qi, especially strengthening the spleen and tonifying the lungs;
With regard to the minister medicine: Salviae Miltiorrhizae Radix et Rhizoma was first recorded in the Divine Farmer's Materia Medica, where it was classified as a top-grade herbal medicine. It is commonly used for activating blood and dissolving stasis. Modern pharmacological studies have demonstrated that tanshinones and salvianolic acids, the ingredients of Salviae Miltiorrhizae Radix et Rhizoma, possess anti-inflammatory properties, inhibit collagen production, promote fibrin degradation, and suppress cellular proliferation. These combined actions underpin their therapeutic potential in preventing and treating tissue and organ fibrosis. Salviae Miltiorrhizae Radix et Rhizoma, Angelicae Sinensis Radix and Chuanxiong Rhizoma are used in combination as the minister medicine and have the effects of activating blood and dissolving stasis;
With regard to the assistant medicine: As recorded in the Divine Farmer's Materia Medica, Platycodonis Radix “is pungent in taste, slightly warm in nature, and attributed to the lung meridian”. It has the functions of dispersing lung Qi and eliminating phlegm. As recorded in the Divine Farmer's Materia Medica, Armeniacae Semen Amarum is better and slightly warm; it has mild toxicity. It is attributed to the lung and large intestine meridians, and functions to depress Qi and relieve cough and asthma. As recorded in the Divine Farmer's Materia Medica, Schisandrae Chinensis Fructus is warm in nature, and it tastes sour and sweet. Schisandrae Chinensis Fructus is attributed to the lung, heart, and kidney meridians. Its principal actions are to replenish Qi, treat cough and rebellious Qi ascent, induce astringency and consolidation, calm cough and expel phlegm, and address chronic cough and dyspnea of deficiency type. Simultaneously, it possesses the effect of promoting the body's immune function. Platycodonis Radix, Armeniacae Semen Amarum and Schisandrae Chinensis Fructus are used in combination as the assistant medicine and have the effects of relieving cough and asthma;
With regard to the guide medicine: Glycyrrhizae Radix et Rhizoma, sweet in taste and neutral in nature, acts to dispel phlegm, relieve cough, tonify the middle Jiao and boost Qi, and harmonize herbal property. Modern pharmacological research has revealed that Glycyrrhizae Radix et Rhizoma contains a variety of chemical ingredients, which have anti-inflammatory, immunomodulatory, and anti-fibrotic effects;
With regard to the whole prescription: Astragali Radix, Codonopsis Radix, Atractylodis Macrocephalae Rhizoma, Poria, Platycodonis Radix, Salviae Miltiorrhizae Radix et Rhizoma, Chuanxiong Rhizoma, Angelicae Sinensis Radix, Armeniacae Semen Amarum, Schisandrae Chinensis Fructus and Glycyrrhizae Radix et Rhizoma together exert the efficacies of strengthening and replenishing Qi (the spleen and the lung), activating blood and dissolving stasis, and relieving cough and asthma.

The present disclosure also provides a preparation method for the above-mentioned traditional Chinese medicine composition, comprising the steps of:
mixing the Astragali Radix, the Codonopsis Radix, the Atractylodis Macrocephalae Rhizoma, the Poria, the Armeniacae Semen Amarum, the Platycodonis Radix, the Salviae Miltiorrhizae Radix et Rhizoma, the Chuanxiong Rhizoma, the Angelicae Sinensis Radix, the Schisandrae Chinensis Fructus and the Glycyrrhizae Radix et Rhizoma to obtain a raw material mixture; mixing the raw material mixture with water and soaking the mixture, decocting the mixture for extraction and filtering the decoction mixture to obtain a filtrate.

In the present disclosure, the raw material mixture of Astragali Radix, Codonopsis Radix, Atractylodis Macrocephalae Rhizoma, Poria, Armeniacae Semen Amarum, Platycodonis Radix, Salviae Miltiorrhizae Radix et Rhizoma, Chuanxiong Rhizoma, Angelicae Sinensis Radix, Schisandrae Chinensis Fructus and Glycyrrhizae Radix et Rhizoma is obtained by mixing the commercially available prepared slices of each raw material. The resulting raw material mixture is mixed with water, soaked, and decocted for extraction, and the decoction mixture is filtered to obtain a filtrate. A mass volume ratio of the raw material mixture to water is preferably 1:6-11, further preferably 1:7-10, more preferably 1:8-9, such as 1:8 or 1:9, and the time for soaking is preferably 0.5-1.5 h, such as 0.5 h, 1 h or 1.5 h; the extraction is performed 1-3 times, such as 1 or 2 times with each extraction lasting preferably 0.5-1.5 h, such as 0.5 h, 1 h or 1.5 h.

In a preferred embodiment, the preparation method for the traditional Chinese medicine composition further comprises the steps of:
subjecting the filtrate to a first concentration to obtain concentrate 1, allowing the concentrate 1 to cool and settle and filtering the mixture to obtain filtrate a, then subjecting the filtrate a to a second concentration and then filtering the resulting mixture to obtain concentrate 2, i.e., the traditional Chinese medicine composition. The first concentration or the second concentration is performed preferably at 60-90° C. under reduced pressure of −0.04 to −0.1 MPa, and further preferably at 70-80° C. under reduced pressure of −0.05 to −0.08 MPa; the concentrate 1 has a relative density of preferably 1.04-1.06, such as 1.04, 1.05 or 1.06; the concentrate 2 has a relative density of preferably 1.02-1.04, such as 1.02, 1.03 or 1.04. The cooling is carried out at a temperature of 10° C. or less. A settling time is preferably 8-24 h, further preferably 10-20 h, and more preferably 14-18 h, such as 14, 15, 16, 17 or 18 h. The filtering can be carried out using a 300-mesh nylon filter cloth.

The present disclosure also provides a traditional Chinese medicine decoction for treating interstitial lung disease, comprising the traditional Chinese medicine composition described above or the traditional Chinese medicine composition obtained by the preparation method described above.

Preferably, the traditional Chinese medicine decoction further comprises 2-4 parts of potassium sorbate.

The present disclosure also provides a preparation method for the above-mentioned traditional Chinese medicine decoction, comprising the steps of:

uniformly mixing the traditional Chinese medicine composition obtained by the preparation method described above, potassium sorbate and water, boiling the mixture and then allowing the mixture to cool down to obtain the traditional Chinese medicine decoction. The cooling is carried out at a temperature of 50° C. or less. Based on a total final volume of 1000 mL for the mixture, the amount of water used is defined as the balance volume required to made up to 1000 mL after dissolving the mixture of the traditional Chinese medicine composition and potassium sorbate.

Through the investigation of modern preparation techniques and the establishment of a multi-index quality control system, the traditional Chinese medicine decoction is prepared in the present disclosure. This results in a compound traditional Chinese medicine preparation process with stable craftsmanship and controllable quality, alongside a quality standard that ensures good patient compliance, thereby laying a foundation for the primary pharmacodynamic study and clinical data collection of the traditional Chinese medicine composition. In addition, through network pharmacology, the active ingredients and target information of the medicament were obtained, and the primary pharmacodynamics of the traditional Chinese medicine decoction were verified in vivo and in vitro. The molecular mechanism of action of the prescription in the treatment of ILD was preliminarily explored. In clinical practice, it is well received by patients for improving clinical symptoms, traditional Chinese medicine syndromes, and improving quality of life. The above studies have confirmed the effectiveness of the traditional Chinese medicine decoction in treating ILD. It has laid a solid foundation for advancing traditional Chinese medicine decoctions towards new traditional Chinese medicaments.

The present disclosure also provides the use of the above-mentioned traditional Chinese medicine composition, the traditional Chinese medicine composition obtained by the preparation method described above, the traditional Chinese medicine decoction described above, or the traditional Chinese medicine decoction obtained by the preparation method described above in the preparation of a medicament for at least one of:
(1) the prevention and/or treatment of interstitial lung disease; and
(2) the modulation of the intestinal flora.

At the phylum level, modulating the intestinal flora according to the present disclosure includes reducing the Firmicutes flora, increasing the Bacteroidota flora, and increasing the ratio of Proteobacteria and Actinobacteriota. At the genus level, modulating the intestinal flora includes modulating the short-chain fatty acid-producing bacterial flora, such as Alloprevotella and unclassified_Muribaculaceae.

On this basis, the present disclosure also provides the use of the above-mentioned traditional Chinese medicine composition, the traditional Chinese medicine composition obtained by the preparation method described above, the traditional Chinese medicine decoction described above, or the traditional Chinese medicine decoction obtained by the preparation method described above in the preparation of a product of the short-chain fatty acid-producing bacterial flora.

In the present disclosure, the short-chain fatty acid-producing bacterial flora includes Alloprevotella and unclassified_Muribaculaceae. The product includes a reagent or a medicament.

Compared with conventional technology, the present disclosure has the following beneficial effects:
The present disclosure provides a traditional Chinese medicine composition and a traditional Chinese medicine decoction for preventing and/or treating interstitial lung disease, and preparation methods therefor and the use thereof. For the traditional Chinese medicine composition, the four ingredients Astragali Radix, Codonopsis Radix, Atractylodis Macrocephalae Rhizoma and Poria are used in combination as the monarch medicine and have the effects of invigorating qi; Salviae Miltiorrhizae Radix et Rhizoma, Angelicae Sinensis Radix and Chuanxiong Rhizoma are used in combination as the minister medicine and have the effects of activating blood and dissolving stasis; the three ingredients Platycodonis Radix, Armeniacae Semen Amarum and Schisandrae Chinensis Fructus are used in combination as the assistant medicine and have the effects of relieving cough and asthma; Glycyrrhizae Radix et Rhizoma is used as the guide medicine and has the effects of dispelling phlegm, relieving cough, tonifying the middle Jiao and boosting Qi, and harmonizing herbal property. These ingredients together have the effects of strengthening and replenishing Qi (the spleen and the lung), activating blood and dissolving stasis, and relieving cough and asthma. The results show that the traditional Chinese medicine composition or traditional Chinese medicine decoction can improve lung function, inhibit pulmonary fibrosis and inflammation, and modulate the intestinal flora, thereby preventing and/or treating interstitial lung disease. The preparation methods for the traditional Chinese medicine composition or traditional Chinese medicine decoction of the present disclosure are simple and easy to operate, and are suitable for industrial production.

### BRIEF DESCRIPTION OF THE DRAWINGS

FIG. 1 shows the effects of the medicated serum, prepared with the traditional Chinese medicine decoction, on Transforming growth factor-β1 (TGF-β1)-induced epithelial-mesenchymal transition (EMT) in A549 cells;

FIG. 2 shows the regulatory effect of the medicated serum, prepared with the traditional Chinese medicine decoction, on the transformation of pulmonary fibroblasts into myofibroblasts induced by TGF-β1;

FIG. 3 shows the effects of the medicated serum, obtained using the traditional Chinese medicine decoction, on the protein levels of E-cadherin and Vimentin in A549 cells as determined by Western blot;

FIG. 4 shows the changes in the lung coefficient of mice after treatment in different groups;

FIG. 5 shows the tidal volume of mice after treatment in different groups;

FIG. 6 shows the minute ventilation of mice after treatment in different groups;

FIG. 7 shows the expiratory flow rate at 50% of the exhaled volume in mice after treatment in different groups;

FIG. 8 shows the HE staining results for lung tissues from mice after treatment in different groups;

FIG. 9 shows the results of Masson's trichrome staining, Sirius red staining, and Van Gieson (VG) staining for lung tissues from mice after treatment in different groups;

FIG. 10 shows the serum levels of IL-1β in mice after treatment in different groups;

FIG. 11 shows the serum levels of IL-6 in mice after treatment in different groups;

FIG. 12 shows the serum levels of TNF-α in mice after treatment in different groups;

FIG. 13 shows the serum levels of TGF-β1 in mice after treatment in different groups;

FIG. 14 shows the Venn diagram after treatment in different groups, where CON represents the blank control group, MOD represents the model group, Nib represents the nintedanib group, FJD represents the low-dose prescription group, and FJG represents the high-dose prescription group;

FIG. 15 shows the diagram of the distribution of OTU numbers after treatment in different groups, where CON represents the blank control group, MOD represents the model group, NIB represents the nintedanib group, FJD represents the low-dose prescription group, and FJG represents the high-dose prescription group;

FIG. 16A-FIG. 16D show the analysis chart of the Alpha diversity index after treatment in different groups, where CON represents the blank control group, MOD represents the model group, NIB represents the nintedanib group, FJD represents the low-dose prescription group, and FJG represents the high-dose prescription group;

FIG. 17A-FIG. 17C show the analysis chart of Beta diversity after treatment in different groups, where CON represents the blank control group, MOD represents the model group, NIB represents the nintedanib group, FJD represents the low-dose prescription group, and FJG represents the high-dose prescription group;

FIG. 18 shows the differences in the bacterial flora in the samples from different groups analyzed at the phylum level, where CON represents the blank control group, MOD represents the model group, NIB represents the nintedanib group, FJD represents the low-dose prescription group, and FJG represents the high-dose prescription group;

FIG. 19 shows the differences in Bacteroidota abundance in the samples from different groups, where CON represents the blank control group, MOD represents the model group, NIB represents the nintedanib group, FJD represents the low-dose prescription group, and FJG represents the high-dose prescription group;

FIG. 20 shows the differences in Firmicutes abundance in the samples from different groups, where CON represents the blank control group, MOD represents the model group, NIB represents the nintedanib group, FJD represents the low-dose prescription group, and FJG represents the high-dose prescription group;

FIG. 21 shows the ratio of Firmicutes to Bacteroidota in the samples from different groups, where CON represents the blank control group, MOD represents the model group, NIB represents the nintedanib group, FJD represents the low-dose prescription group, and FJG represents the high-dose prescription group;

FIG. 22 shows the differences in the bacterial flora in the samples from different groups analyzed at the genus level, where CON represents the blank control group, MOD represents the model group, NIB represents the nintedanib group, FJD represents the low-dose prescription group, and FJG represents the high-dose prescription group;

FIG. 23 shows the differences in the abundance of Alloprevotella, a short-chain fatty acid-producing bacterium, in the samples from different groups, where CON represents the blank control group, MOD represents the model group, NIB represents the nintedanib group, FJD represents the low-dose prescription group, and FJG represents the high-dose prescription group;

FIG. 24 shows the differences in the abundance of unclassified_Muribaculaceae, a short-chain fatty acid-producing bacterial flora, in the samples from different groups.

### DETAILED DESCRIPTION OF THE EMBODIMENTS

In the present disclosure, unless otherwise specified, all raw material components are commercially available and well known to those skilled in the art.

The technical solutions provided by the present disclosure will be described in detail in conjunction with the examples below, which cannot be construed as limiting the scope of protection of the present disclosure.

In the Examples below, all the raw materials are used in the form of prepared slices.

### Example 1

A traditional Chinese medicine composition for preventing and/or treating interstitial lung disease was prepared from the following raw materials in the amounts:

30 g of Astragali Radix, 20 g of Codonopsis Radix, 12 g of Atractylodis Macrocephalae Rhizoma, 15 g of Poria, 12 g of Armeniacae Semen Amarum, 12 g of Platycodonis Radix, 15 g of Salviae Miltiorrhizae Radix et Rhizoma, 15 g of Chuanxiong Rhizoma, 12 g of Angelicae Sinensis Radix, 6 g of Schisandrae Chinensis Fructus, and 9 g of Glycyrrhizae Radix et Rhizoma.

The traditional Chinese medicine composition was obtained by the preparation method comprising the steps of:
(1) Prescription herbs that met the quality standards in Volume I (Prepared Slices) of the Chinese Pharmacopoeia (2020 Edition) and passed qualification testing were used. Astragali Radix, Codonopsis Radix, Atractylodis Macrocephalae Rhizoma, Poria, Armeniacae Semen Amarum, Platycodonis Radix, Salviae Miltiorrhizae Radix et Rhizoma, Chuanxiong Rhizoma, Angelicae Sinensis Radix, Schisandrae Chinensis Fructus and Glycyrrhizae Radix et Rhizoma in the above-mentioned prescription amounts were weighed and mixed to obtain a prepared slice mixture;
(2) The prepared slice mixture was soaked in 1442 mL (a 9-fold volume) of water for 1 h, decocted for 1 h, and then filtered through a 300-mesh nylon cloth to obtain a first filtrate and the drug residue. The drug residue was soaked in another 1264 mL (an 8-fold volume) of water, decocted for 1 h, and filtered through a 300-mesh nylon cloth. The drug residue was discarded, and a second filtrate was obtained. The resulting first filtrate and second filtrate were separately concentrated under reduced pressure of −0.04 to −0.1 MPa at 60-90° C. until the relative density of the concentrates reached 1.04-1.06 at 50-60° C. The first concentrate and the second concentrate were combined to obtain concentrate 1;
(3) Concentrate 1 was cooled to below 10° C. and allowed to stand. After 16 h, supernatant 1 was collected by filtration through a 300-mesh nylon cloth. The precipitate solution was left to stand for another 16 h. Supernatant 2 was then collected by filtration through a 300-mesh nylon cloth. Supernatant 1 and supernatant 2 were combined to obtain filtrate a. Filtrate a was concentrated under reduced pressure of −0.04 to −0.1 MPa at 60-90° C. until the relative density of the concentrate reached 1.02-1.04 at 50-60° C. The resulting concentrate was filtered through a 300-mesh nylon cloth to obtain concentrate 2, which was the final traditional Chinese medicine composition.

### Example 2

A traditional Chinese medicine decoction for preventing and/or treating interstitial lung disease consists of the following raw materials in the amounts:

30 g of Astragali Radix, 20 g of Codonopsis Radix, 12 g of Atractylodis Macrocephalae Rhizoma, 15 g of Poria, 12 g of Armeniacae Semen Amarum, 12 g of Platycodonis Radix, 15 g of Salviae Miltiorrhizae Radix et Rhizoma, 15 g of Chuanxiong Rhizoma, 12 g of Angelicae Sinensis Radix, 6 g of Schisandrae Chinensis Fructus, 9 g of Glycyrrhizae Radix et Rhizoma and 3 g of potassium sorbate and pure water.

The traditional Chinese medicine decoction was obtained by the preparation method comprising the steps of:

The preparation method for the traditional Chinese medicine composition was the same as that described in Example 1.

Potassium sorbate in the above-mentioned amount was added to concentrate 2, and the volume was made up to 1000 mL with water. The mixture was stirred and mixed, boiled for 20 min, and cooled to below 50° C. to obtain a traditional Chinese medicine decoction.

[Description]: The traditional Chinese medicine decoction was a dark brown liquid with a slight fragrance, and a bitter yet slightly sweet taste.

### Identification

### (1) Identification of Atractylodis Macrocephalae Rhizoma by TLC

The traditional Chinese medicine decoction (20 mL) was taken and extracted with n-hexane (2×25 mL). The n-hexane layers were combined and evaporated to dryness. The residue was dissolved in 2 mL of methanol to obtain a test sample solution. Separately, 1 g of the reference crude drug of Atractylodis Macrocephalae Rhizoma was taken. n-hexane (10 mL) was added. The mixture was ultrasonicated for 30 minutes and then filtered. The filtrate was evaporated to dryness. The residue was dissolved in 2 mL of methanol to obtain a solution as the reference crude drug solution of Atractylodis Macrocephalae Rhizoma. The test was performed in accordance with the Thin Layer Chromatography (General Rule 0502 of the Chinese Pharmacopoeia, 2020 Edition). The two aforementioned solutions (10 μL each) were separately taken and applied to the same silica gel G thin layer plate. A developing solvent, which was a mixture of petroleum ether (60-90° C.)-ethyl acetate (50:1), was used for development. After development, the plate was removed and dried in air. It was then sprayed with a 5% vanillin in sulfuric acid solution. The plate was heated at 105° C. until the color of the spots became distinct. The plate was examined under daylight.

Results: a spot of the same color appeared at the same position in the chromatogram of the test sample as in the chromatogram of the reference crude drug.

### (2) Identification of Poria by TLC

The traditional Chinese medicine decoction (20 mL) was taken and extracted with diethyl ether (2×30 mL). The diethyl ether layers were combined and evaporated to dryness. The residue was dissolved in 2 mL of n-hexane to obtain a test sample solution. Separately, 1 g of the reference crude drug of Poria was taken. Diethyl ether (10 mL) was added. The mixture was ultrasonicated for 30 minutes and then filtered. The filtrate was evaporated to dryness. The residue was dissolved in 2 mL of n-hexane to obtain a solution as the reference crude drug solution of Poria. The test was performed in accordance with the Thin Layer Chromatography (General Rule 0502 of the Chinese Pharmacopoeia, 2020 Edition). The two aforementioned solutions (5 μL each) were separately taken and applied to the same silica gel G thin layer plate. A developing solvent, which was a mixture of petroleum ether (30-60° C.)-ethyl acetate-acetone (84:3:15), was used for development. After development, the plate was removed and dried in air. It was then sprayed with a 5% vanillin in sulfuric acid solution. The plate was heated at 105° C. until the color of the spots became distinct. The plate was examined under an ultraviolet lamp (365 nm).

Results: a spot of the same color appeared at the same position in the chromatogram of the test sample as in the chromatogram of the reference crude drug.

### (3) Identification of Platycodonis Radix by TLC

The traditional Chinese medicine decoction (30 mL) was taken and evaporated to dryness. 50 mL of 7% sulfuric acid in ethanol-water (1:3) were added to the residue. The mixture was heated under reflux for 3 hours and then extracted with diethyl ether (2×30 mL). The diethyl ether layers were combined and evaporated to dryness. The residue was dissolved in 2 mL of n-hexane to obtain a test sample solution. Separately, 1 g of the reference crude drug of Poria was taken. Diethyl ether (10 mL) was added. The mixture was ultrasonicated for 30 minutes and then filtered. The filtrate was evaporated to dryness. The residue was dissolved in 2 mL of n-hexane to obtain a solution as the reference crude drug solution of Poria. The test was performed in accordance with the Thin Layer Chromatography (General Rule 0502 of the Chinese Pharmacopoeia, 2020 Edition). The two aforementioned solutions (5 μL each) were separately taken and applied to the same silica gel G thin layer plate. A developing solvent, which was a mixture of petroleum ether (30-60° C.)-ethyl acetate-acetone (84:3:15), was used for development. After development, the plate was removed and dried in air. It was then sprayed with a 5% vanillin in sulfuric acid solution. The plate was heated at 105° C. until the color of the spots became distinct. The plate was examined under an ultraviolet lamp (365 nm).

Results: a spot of the same color appeared at the same position in the chromatogram of the test sample as in the chromatogram of the reference crude drug.

### Content Determination

Astragaloside IV was determined by the high-performance liquid chromatography (General Chapter 0512 of Chinese Pharmacopoeia (2020 Edition)).

Chromatographic conditions and system suitability test Octadecylsilane-bonded silica gel was used as the filler. Acetonitrile-water (36:64) was used as the mobile phase; An evaporative light-scattering detector was used for detection. The number of theoretical plates, which was calculated based on the peak of astragaloside IV, should not be less than 4000.

Preparation of reference solution: an appropriate amount of a reference of Astragaloside IV was taken and precisely weighed and methanol was added to prepare a 1 mL solution containing Astragaloside IV (0.2 mg), which was the reference solution.

Preparation of test sample solution: the traditional Chinese medicine decoction was mixed uniformly. The decoction (20 mL) was taken and extracted under shaking with water-saturated n-butanol (4×20 mL). The n-butanol layers were combined and washed with ammonia test solution (2×40 mL). The ammonia test solutions were discarded. The n-butanol solution was evaporated to dryness. The residue was dissolved in methanol and then transferred to a 5-mL volumetric flask. Methanol was added to the flask until the volume reached the given calibration mark. The solution in the flask was mixed well and then filtered. The subsequent filtrate was collected to obtain the test sample solution.

Assay: the reference solution and the test sample solution (10 μL each) were each aspirated precisely and injected into the liquid chromatograph to determine the contents.

Results: Each 1 mL of the preparation contains not less than 0.15 mg of Astragali Radix (calculated based on astragaloside IV (C₄₁H₆₈O₁₄)).

[Administration and dosage] oral administration, 30 mL each time, three times a day, shaken well before use.

[Actions and indications] The traditional Chinese medicine decoction possesses the effects of strengthening the spleen and tonifying the lungs, activating blood and dissolving stasis, dispelling phlegm and unblocking the collaterals. It is indicated for patients with lung impediment attributed to Qi deficiency, blood stasis, and phlegm obstruction. The common clinical manifestations include dyspnea, wheezing and fatigue, cough with or without sputum, dark red tongue with a thin white coating, and thin weak pulse. In terms of Western medicine system, it is used for conditions such as interstitial pneumonia or interstitial lung disease.

### Example 3

### Pharmacodynamic Study of Traditional Chinese Medicine Decoction

### 3.1 Cell Experiment: Study on Effect of Traditional Chinese Medicine Decoction on TGF-β1-Induced Mesenchymal Transition in A549 Cells

A549 cells were cultured in vitro, induced with TGF-β1 and then intervened with the traditional Chinese medicine decoction. The progression of fibrosis was assessed by measuring the expression of epithelial-mesenchymal transition markers. The effects of the traditional Chinese medicine decoction on inhibiting the TGF-β1-induced transdifferentiation of A549 cells were elucidated by detecting the expression of fibrosis markers, oxidative stress indicators, and inflammatory cytokines.

### 3.1.1. Study Methods

### (1) Selection of Cells

A549 cell is a human lung adenocarcinoma epithelial cell. It exhibits characteristics of type II alveolar epithelial cells during in vitro culture. It is currently a cellular model commonly used for evaluating epithelial-mesenchymal transition (EMT).

### (2) Preparation of Traditional Chinese Medicine Decoction

The traditional Chinese medicine decoction was prepared by the preparation method for the traditional Chinese medicine decoction in Example 2.

### (3) Preparation of Rat Medicated Serum

Six-week-old SD rats were fed adaptively for one week. The rats then received the traditional Chinese medicine decoction by gavage for 7 consecutive days (8.1 mL each time, once daily). After the last dose, the rats were fasted for 9 h but had free access to water. Then the rats were anesthetized by an intraperitoneal injection of 1% pentobarbital sodium (50 mg/kg). Blood was collected from the abdominal aorta. The blood samples were allowed to stand for 1 h and then centrifuged at 4000×g for 10 minutes. The serum was collected, aliquoted, and labeled to obtain the medicated serum. The medicated serum was stored at −80° C. for later use.

Another 10 SD rats received an equivalent amount of physiological saline by gavage. Blank serum was prepared following the same procedure and stored for later use.

Both the medicated serum and the blank serum were inactivated at 56° C. for 30 min, then filtered and sterilized prior to use.

### (4) Model Establishment and Drug Treatment

Current research has found that alveolar epithelial-mesenchymal transition (EMT) is critical in the formation of fibrosis. Transforming growth factor-(31 (TGF-β1) is the master switch of the EMT process. It can promote the proliferation and differentiation of mesenchymal cells and facilitate the deposition of extracellular matrix (ECM). Therefore, it is considered the most important pro-fibrotic factor and can be used to induce cells to establish a fibrosis model.

The human alveolar epithelial adenocarcinoma cell line A549 was maintained in DMEM medium supplemented with 10% fetal bovine serum (FBS) and 1% penicillin-streptomycin in a CO₂ incubator at 37° C. with 5% CO₂.

Fibrosis model establishment and traditional Chinese medicine decoction treatment: A549 cells were digested with trypsin and seeded into a 6-well plate and cultured overnight in DMEM containing 10% FBS at 37° C. in a 5% CO₂ incubator. The cells were then treated with 0, 5, and 10 ng/mL TGF-β1 for 24 h, respectively. Meanwhile, to determine the effect of the traditional Chinese medicine decoction on A549 cells, a blank serum control group was established. This group was cultured in a medium containing 10% rat blank serum for 48 h. The medicated serum group was cultured in a medium containing 10% rat medicated serum for 24 h. Then, a medium containing 10 ng/mL TGF-β1 (along with 10% rat medicated serum) was added, and the treatment continued for another 24 h. The model group was treated with a medium containing 10% rat blank serum for 24 h. Then, a medium containing 10 ng/mL TGF-β1 (along with 10% rat blank serum) was added, and the treatment continued for another 24 h. After the treatment, the morphology of A549 cells was observed under an inverted optical microscope. The cell culture supernatant and the cells were collected.

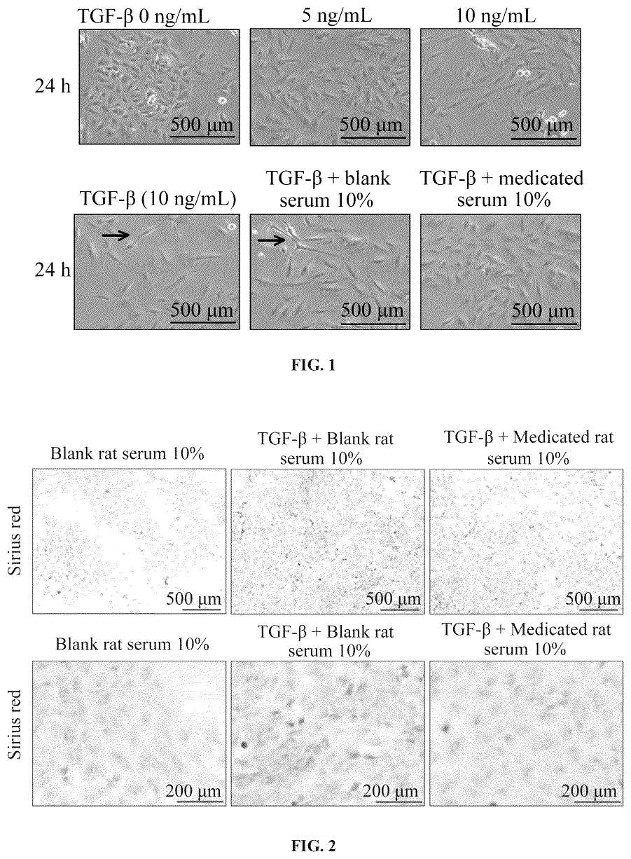

As shown in FIG. 1, stimulation with 10 ng/mL TGF-β1 for 24 h induced a clear morphological shift in A549 cells compared to the control group. The cells transitioned from a cobblestone-like, polygonal epithelial morphology to a spindle-like, elongated mesenchymal morphology, characterized by increased intercellular space and loosened intercellular connections, presenting a fibroblast-like phenotype. Meanwhile, compared with the model group and the blank serum control group, the medicated serum group alleviated the TGF-β1-induced changes in cell morphology.

### (5) Sirius Red Staining

Collagen fiber is the most widely distributed and abundant fiber in connective tissue, and it is extensively distributed in various organs. Type I collagen fiber is found predominantly in bone, skin, and tendons; type II collagen fiber is primarily cartilage collagen; type III collagen fiber is mainly located in embryonic tissues, adult blood vessels, and the gastrointestinal tract; and type IV collagen fiber is primarily located in the basement membrane. Both Sirius red and its counterstain are strongly acidic dyes that readily bind to the basic groups in collagen molecules through firm adsorption. Under polarized light microscopy, collagen fibers have the property of positive uniaxial birefringence. After binding with the Sirius red complex staining solution, the birefringence is enhanced, and the resolution is improved, thereby allowing differentiation between different types of collagen fibers.

In this example, the modified Sirius red staining solution (for collagen fiber staining), G1472-reagent B, purchased from Beijing Solarbio Science & Technology Co., Ltd. was used. Under a conventional optical microscope, collagen fibers in tissues such as heart and blood vessels are stained red. Under polarized light microscopy, Sirius red staining provides assistance for the typing and grading studies of various fibrotic lesions. The expression of collagen in cells was preliminarily determined by observing the changes in the concentration of Sirius red staining.

In the present disclosure, the morphological changes of A549 cells after treatment with different concentrations of TGF-β1 were observed using an inverted microscope. The status of collagen fibers was observed with the aid of Sirius red staining to establish an in vitro pulmonary fibrosis model. The inhibitory effect of the medicated serum on TGF-β1-induced EMT in A549 cells was detected.

The results in FIG. 2 showed that after 24 h of TGF-β1 stimulation, collagen fiber expression in A549 cells increased significantly. Meanwhile, the medicated serum group inhibited the TGF-β1-induced changes in cell morphology and reduced collagen fiber content within the cells compared with the model group and the blank serum control group.

### (6) Detection by Western Blot Test

Following lysis with RIPA buffer to extract total protein from cell samples of each group, the protein samples were separated by sodium dodecyl sulfate-polyacrylamide gel electrophoresis (SDS-PAGE) and transferred onto PVDF membranes. The membranes were blocked in 5% skim milk/Tris-buffered saline with Tween-20 (TBST) at 37° C. for 2 h. The membranes were rinsed three times with Tris-buffered saline containing 0.1% Tween-20 (TBST), and each rinse lasted for 10 min. The membranes were incubated for 2 h with primary antibodies against GAPDH (diluted at 1:2500) and fibrosis-related proteins, including E-cadherin and Vimentin. The membranes were rinsed three times with TBST, and each rinse lasted for 10 min. The membranes were incubated with horseradish peroxidase-conjugated secondary antibodies (diluted at 1:2000) at 37° C. for 1-2 h. The membranes were washed three times with TBST, and each wash lasted for 10 min. Following visualization with an enhanced chemiluminescence substrate kit, the protein bands were detected with a BIO-RAD ChemiDoc XRS+ chemiluminescence gel imaging system. The grayscale values of the bands of reference protein GAPDH and the fibrosis-related proteins were analyzed using ImageJ software, and the expression level of each target protein was normalized to that of the reference protein GAPDH.

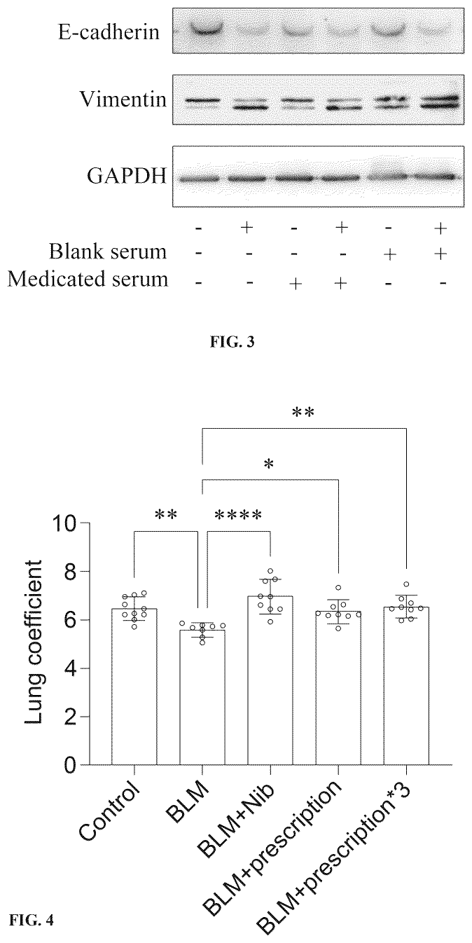

As shown in FIG. 3, TGF-β1 stimulation decreased E-cadherin expression and increased Vimentin expression in A549 cells compared with the blank control group. Conversely, treatment with the medicated serum, obtained using the traditional Chinese medicine decoction, up-regulated E-cadherin and down-regulated Vimentin expression.

In summary, these results indicate that the decoction for tonifying the lungs and freeing bi has the effect of reversing the EMT process in A549 cells.

### 3.2 Animal Experiment: Protective Effect of Traditional Chinese Medicine Decoction on Bleomycin-Induced Pulmonary Fibrosis in Mice

In this example, a mouse model of pulmonary fibrosis induced by bleomycin was established. An intervention study was conducted using the traditional Chinese medicine decoction and nintedanib. The effects of the traditional Chinese medicine decoction on bleomycin-induced pulmonary inflammation and fibrosis in mice were observed. The study aimed to investigate the mechanism of action from perspectives, such as inflammation, macrophage polarization, epithelial-mesenchymal transition, and intestinal flora.

### 3.2.1 Research Methods

### 3.2.2 (1) Selection of Model-Inducing Agent

Bleomycin is an aminoglycoside drug. It is commonly used in the clinical treatment of various malignant tumors. However, its most serious side effect is pulmonary fibrosis. Based on this side effect, it is often used to induce animal models of pulmonary fibrosis in order to study its mechanisms and screen anti-fibrosis medications. Common administration methods include a single intratracheal injection, or intravenous injection, nasal drip, and intraperitoneal injection. The most commonly used and classic administration method for the pulmonary fibrosis model is the single intratracheal injection of bleomycin. This method can establish an animal model with a pathological process similar to that of human interstitial pulmonary fibrosis. Furthermore, the modeling time is short. Fibrosis generally occurs within 14 to 28 days.

### (2) Experimental Mice

Thirty C57BL/6 male mice (body weight range: 18-20 g, 6-8 weeks old) were used. The mice were purchased from Hunan Silaike Jingda Experimental Animal Co., Ltd. with the animal production license number of SCXK (Xiang) 2019-0004.

The mice were maintained in a suitable breeding environment. The ambient temperature was 20-25° C., and the relative humidity was 40%-70%. The noise level was below 85 decibels, and the ammonia concentration was below 20 ppm. Both the mouse bedding and feed were both SPF-grade. The mice had free access to water and diet daily. All animal experimental procedures were performed according to standard protocols. These procedures were reviewed and approved by the Animal Welfare and Ethics Committee of the Chongqing Academy of Chinese Materia Medica.

### (3) Establishment of Bleomycin-Induced Pulmonary Fibrosis Mouse Model

After one week of acclimatization, the mice were randomly divided into groups. The groups were as follows: the blank control (Control) group (n=10), the model (BLM) group (n=12), the nintedanib (BLM+Nib) group (n=12), the low-dose prescription (BLM+prescription) group (n=12), and the high-dose prescription (BLM+prescription*3) group (n=12). The mice were anesthetized by an intraperitoneal injection of 1% pentobarbital sodium (50 mg/kg). The mice in the model group, the nintedanib group, the low-dose prescription group, and the high-dose prescription group were slowly administered a 3 mg/kg bleomycin solution using a microsyringe. Immediately after the injection, the mice were held upright and rotated to ensure even distribution of the drug solution in the lungs. The mice in the blank control group were slowly administered physiological saline using a microsyringe. At hour 48 after model induction, drug administration began. The blank control group and the model group received an equivalent amount of physiological saline by gavage. The nintedanib (BLM+Nib) group received nintedanib esylate by gavage (22.75 mg/kg each administration, twice a day, with 12-h intervals between administrations). The low-dose prescription group received the traditional Chinese medicine decoction prepared in Example 2 by gavage (20.54 g/kg/d, once daily). The high-dose prescription group received the traditional Chinese medicine decoction prepared in Example 2 by gavage (61.62 g/kg/d, once daily). These doses corresponded to 1-fold and 3-fold of the recommended human daily dose, respectively.

The drug administration continued for 28 consecutive days. The fur color and diet of the mice were observed and their body weight was measured every day. On day 28, the mice were anesthetized by an intraperitoneal injection of 1% pentobarbital sodium (50 mg/kg). The mice were sacrificed by drawing blood from the abdominal aorta. The lung tissues were immediately dissected and rinsed with cold physiological saline. After the moisture was blotted dry with filter paper, the wet weight of the lungs was measured. The same lobes of the right lungs were placed in 4% paraformaldehyde solution. The remaining lung tissues were collected and stored at −80° C. for subsequent experimental studies.

After one week of acclimatization, the male C57BL/6 mice showed a good mental state, normal breathing, smooth fur, well-developed muscles, and high activity levels. Their eyes were bright and alert, and their responses were quick. Their limbs were symmetrical, with no physical deformities or wounds. There were no abnormal secretions from the natural orifices. The mice in the blank control group appeared normal throughout the experimental period. After model induction, the mice in the model group displayed ruffled fur and lethargy. The symptoms of the mice in the drug treatment groups showed improvement.

### (4) Lung Coefficient Calculation

The lung coefficients of various groups of mice were calculated according to the following formula.

Lung coefficient=Lung mass (g)/Body mass (kg).

The lung coefficient results of various groups are shown in FIG. 4. The results in FIG. 4 showed that the lung coefficients of the model mice decreased significantly. This finding indicated the successful establishment of the models. The lung coefficients of the mice in all drug treatment groups increased. These results suggested that the drugs may have the effects of improving lung function in mice.

### (5) Detection of Lung Function in Mice

Three days before the end of the experiment, the lung functions of mice in various groups were detected using unrestrained whole body plethysmography (WBP) system from Shanghai TOW Intelligent Technology Co., Ltd. The various indicators and their meanings are detailed in the result analysis section. Airway pressure, lung volume, and flow rate are the three essential elements of lung function measurement. The basic working principle of WBP is Boyle's law, which states that the pressure and volume of a fixed amount of gas in a sealed container are inversely proportional at a constant temperature.

The plethysmography chamber of the WBP system is equivalent to a sealed container. The animal is placed inside the chamber. When the animal inhales, the temperature and relative humidity of the inhaled gas increase because the respiratory system's temperature and humidity are higher than those of the inhaled gas. The mixed gas expands, and the animal's thoracic cavity expands, causing the pressure inside the chamber to increase. A larger inhaled gas volume (tidal volume) results in a more significant pressure increase inside the chamber. Conversely, when the animal exhales, the exhaled gas mixes with the air. As the air temperature and humidity decrease, the gas is compressed, and the pressure inside the chamber decreases. The pressure changes (ΔP) in the plethysmography chamber caused by the animal's breathing are recorded by a screen-type respiratory sensor within the chamber and then amplified by an amplifier. These pressure changes can be used to derive other lung function indicators using specialized algorithms in the analysis software.

The WBP system was used to collect the following measurement indicators: tidal volume (the volume of gas inhaled or exhaled during quiet breathing), minute ventilation (the total volume of gas entering or leaving the lungs per minute), and mid-expiratory flow rate (the expiratory flow rate at 50% of the exhaled tidal volume).

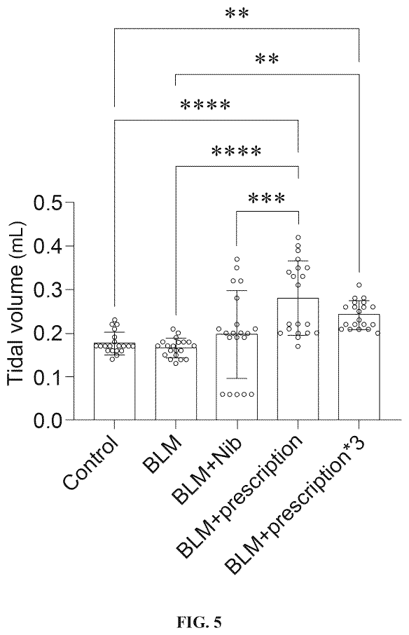

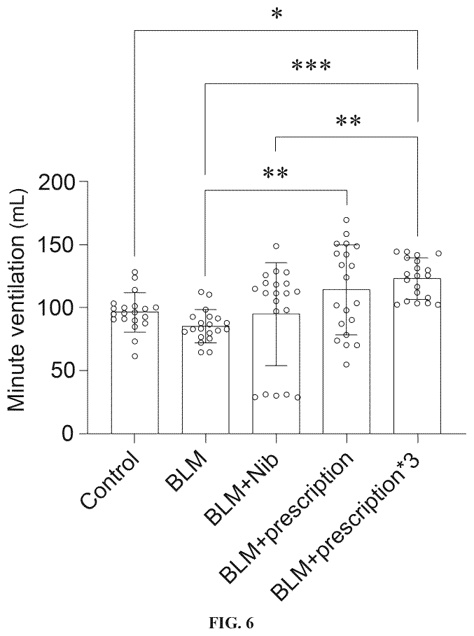

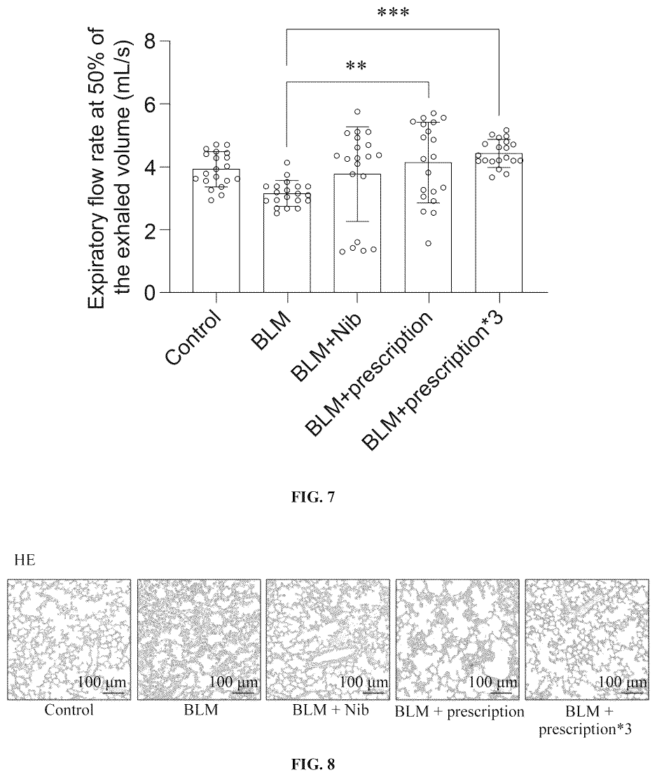

The results in FIGS. 5-7 showed that the medicament increased the tidal volume, minute ventilation and expiratory flow rate at 50% of the exhaled tidal volume in mice, suggesting an improvement in pulmonary ventilation function and a reduction in airway obstruction.

### (6) Pathological Observation of Lung Tissue

The lung tissues dissected from the mice in various groups were fixed overnight in 4% paraformaldehyde. Then different concentrations of alcohol were used as dehydrating agents. The lung tissues were dehydrated sequentially with alcohol at increasing concentrations to gradually remove water from the tissues. Subsequently, the lung tissues were cleared with xylene. The lung tissues were embedded in paraffin. After the paraffin blocks solidified, the lung tissues were sectioned into 5 μM thin slices using a microtome. The mouse lung tissues were subjected to hematoxylin-eosin (HE) staining. The results were observed under an optical microscope to assess pulmonary inflammation.

The mouse lung tissues were subjected to Masson's trichrome, Sirius red, and Van Gieson (VG) staining. The results were observed under an optical microscope to assess lung status and collagen deposition.

The results in FIG. 8 showed that the alveolar structure of the mice in the blank control group was clear. The lung tissue showed no significant alveolar inflammatory exudation or fibrotic lesions. In the model group, the mice's lung structures became distorted. The number of interstitial cells increased, fibrous tissue proliferated, and fibrosis occurred. Additionally, the alveolar spaces enlarged and fused. After the intervention with Nib and the traditional Chinese medicine decoction, the pathological severity of the lung tissue in the lung fibrosis mice was reduced in the nintedanib group, the low-dose prescription group, and the high-dose prescription group. The structural integrity of the lung tissue was improved. The thickness of the alveolar septum was decreased. The infiltration of inflammatory cells was reduced.

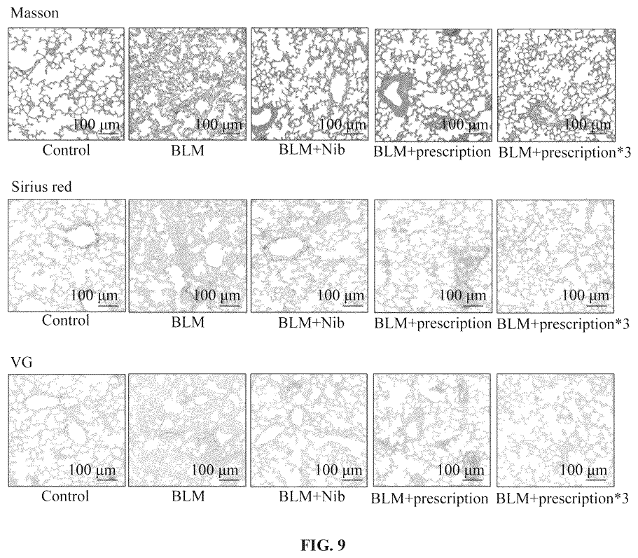

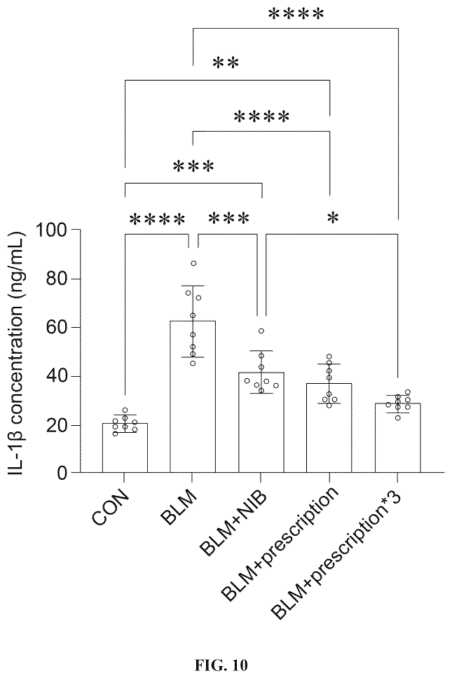

The results in FIG. 9 showed that the lung tissue structure and morphology of the mice in the blank control group were normal. The mice in the model group exhibited severe pulmonary fibrosis, characterized by prominent collagen deposition in the lung interstitium that presented as large bundles and sheets. Following treatment with nib or the traditional Chinese medicine decoction, a reduction in lung collagen deposition and a significant alleviation of pulmonary fibrosis were observed in the nintedanib, low-dose prescription, and high-dose prescription groups.

### (7) Cytokine Detection

Blood was collected and serum was separated. The levels of TNF-α, IL-1β, IL-6, and TGF-β1 in the mouse serum were detected using ELISA kits provided by Shanghai Enzyme-linked Biotechnology Co., Ltd.

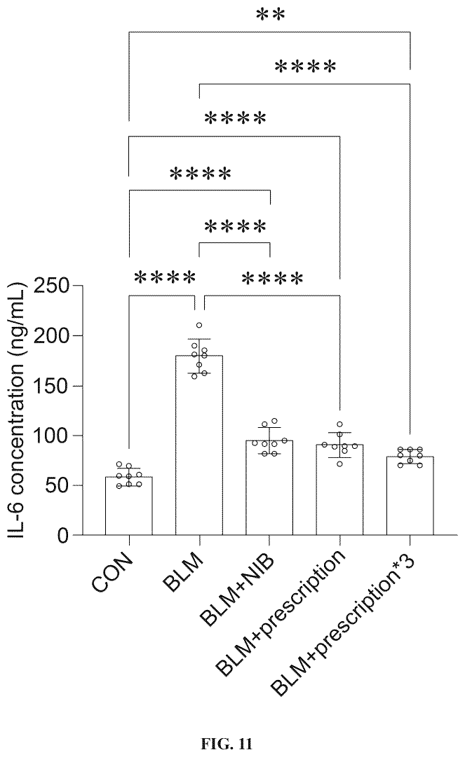

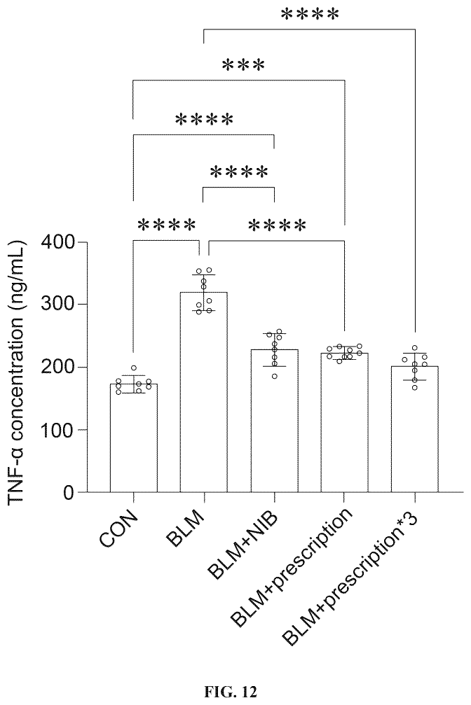

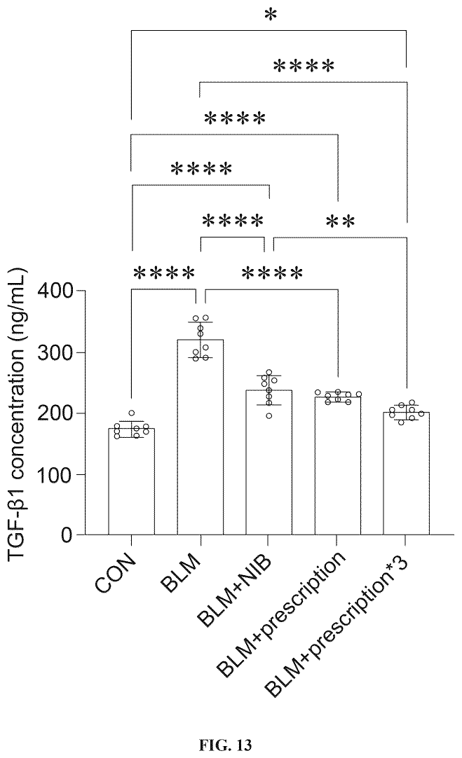

The results in FIGS. 10-13 showed that in the low-dose prescription group and the high-dose prescription group, after the intervention with the traditional Chinese medicine decoction, the level of inflammation was reduced, indicating that the medicament had the effect of regulating the immune response.

### (8) 16S Sequencing of Intestinal Flora

Currently, research on microbial diversity is primarily conducted on the conserved regions of nucleic acid sequences encoding ribosomal RNA. For bacteria, this primarily targets the 16S rDNA, a DNA sequence that encodes prokaryotic small-subunit rRNA (16S rRNA). The species composition of a sample can be revealed by performing reads merging, filtering, clustering, or denoising, followed by species annotation and abundance analysis. Further analyses, such as Alpha Diversity analysis, Beta Diversity analysis, significant species difference analysis, correlation analysis, and functional prediction analysis, can uncover the differences between samples. In this study, 16S rRNA gene sequencing was used to detect and analyze the diversity of the fecal gut microbiome. The workflow was as follows: extraction of fecal DNA via the CTAB method; amplicon generation; mixing and purification of PCR products; library construction and sequencing. Microbial diversity analysis was conducted on the Illumina Novaseq sequencing platform via the paired-end sequencing approach, with small-fragment libraries constructed.

The steps for extracting fecal DNA were as follows: (1) Lysis: 1 mL of CTAB lysis buffer was aspirated into a 2.0 mL EP tube. Subsequently, lysozyme was added, followed by the addition of an appropriate amount of fecal sample. It was then placed in a 65° C. water bath and inverted to mix thoroughly for complete sample lysis; (2) Centrifugation: A mixture of phenol (pH 8.0):chloroform:isoamyl alcohol (25:24:1) was added. The tube was inverted to mix thoroughly and then the mixture was centrifuged for 10 min. The supernatant was collected. A mixture of chloroform:isoamyl alcohol (24:1) was added. The tube was inverted to mix thoroughly and then the mixture was centrifuged for 10 min; (3) Precipitation: The supernatant was aspirated into a 1.5 mL centrifuge tube. Isopropanol was added. The tube was inverted several times and placed at −20° C. for precipitation; (4) Washing: The tube was centrifuged for 10 min. The liquid was decanted, and care was taken not to pour out the precipitate. The precipitate was washed twice with 1 mL of 75% ethanol. Any remaining small amount of liquid can be collected by brief re-centrifugation and aspirated with a pipette tip; (5) Drying: The sample was air-dried at room temperature; (6) The DNA sample was dissolved in DEPC-treated water. To facilitate complete dissolution, the sample was incubated at 55-60° C. for 10 min when necessary.

### (7) Digestion: 1 μL of RNase A was Added to Digest RNA and the Mixture was Placed at 37° C. For 15 Min.

Library construction and sequencing: following total DNA extraction from the sample, primers were designed based on the conserved regions (upstream primer 338F: 5′-ACTCCTACGGGAGGCAGCAG-3′ (SEQ ID NO: 1) and downstream primer 806R: 5′-GGACTACHVGGGTWTCTAAT-3′ (SEQ ID NO: 2)). The bacterial 16S V3-V4 regions, which serve as the target region of the primers, were amplified. The sequencing adapters were ligated to the ends of the primers, and PCR amplification was performed, followed by purification, quantification, and normalization of the PCR products. The constructed library first underwent quality control. Libraries that passed quality control were sequenced using the Illumina Novaseq 6000. Raw image data files obtained from high-throughput sequencing platforms (such as Illumina Novaseq) were converted into raw sequenced reads through base calling analysis. The results were stored in the FASTQ file format (abbreviated as fq), which contains both the sequence information of the reads and their corresponding sequencing quality information. In the sequence as shown in SEQ ID NO: 2, H, V, and W represent degenerate bases, where H=A/C/T; V=A/C/G; W=A/T.

Data preprocessing: (1) Quality filtering: Trimmomatic v0.33 software was first used to filter the raw reads obtained from sequencing. Primer sequences were then identified and trimmed from the reads using software cutadapt 1.9.1, generating primer-free clean reads; (2) Paired-end sequence merging: The clean reads from each sample were merged based on overlap using Usearch (v10). Then, length filtering was applied to the merged data according to the expected length range for each region. (3) Chimera removal: UCHIME (v4.2) was used to identify and remove chimeric sequences, yielding the final effective reads. The BMK Cloud platform tools were used for microbial diversity analysis in this project.

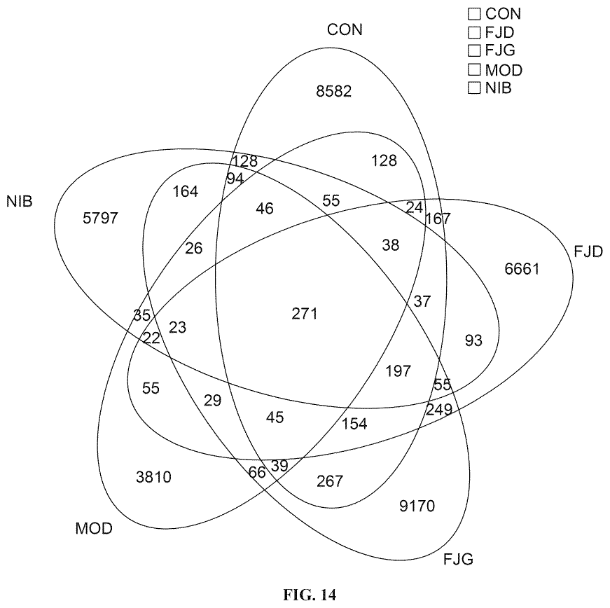

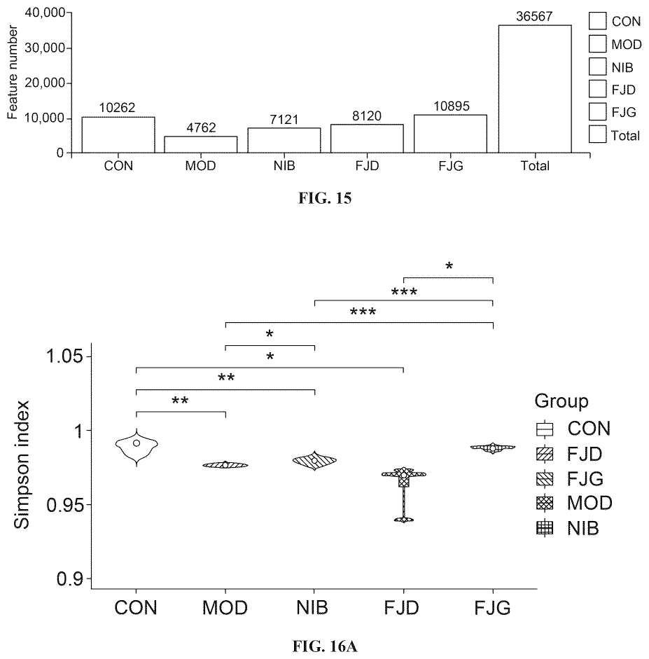

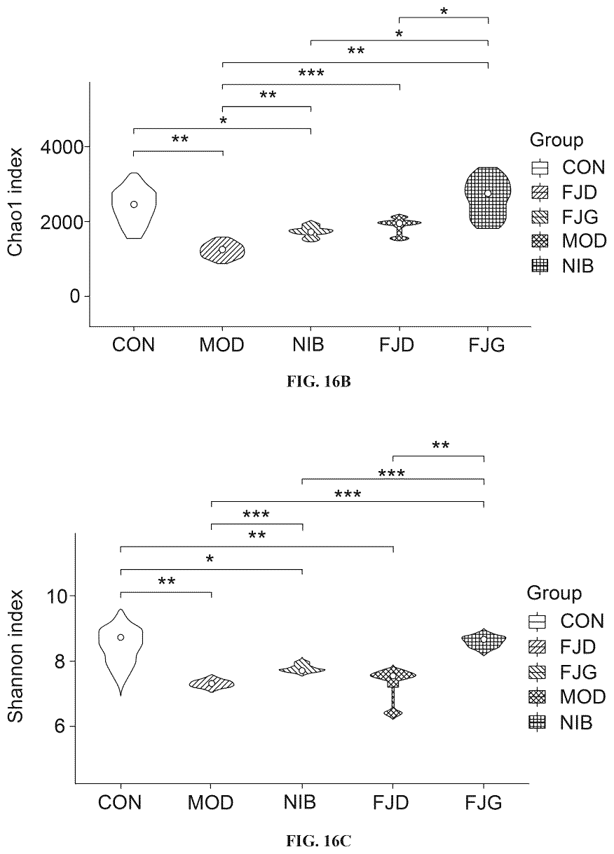

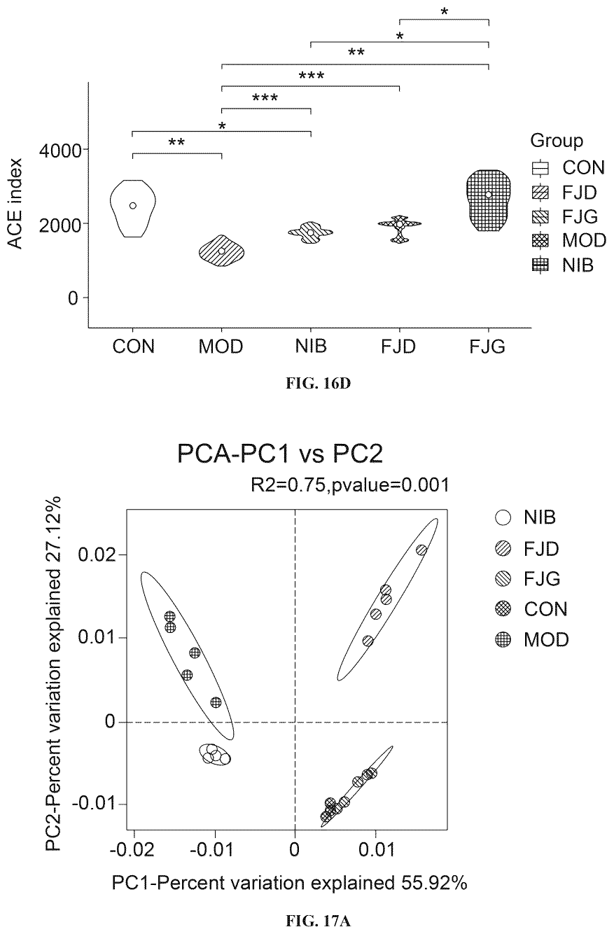

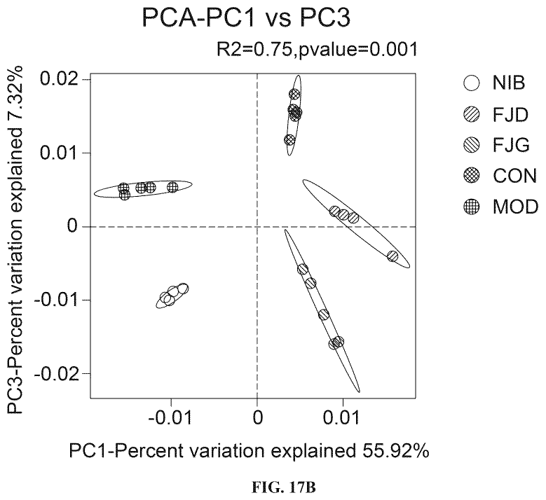

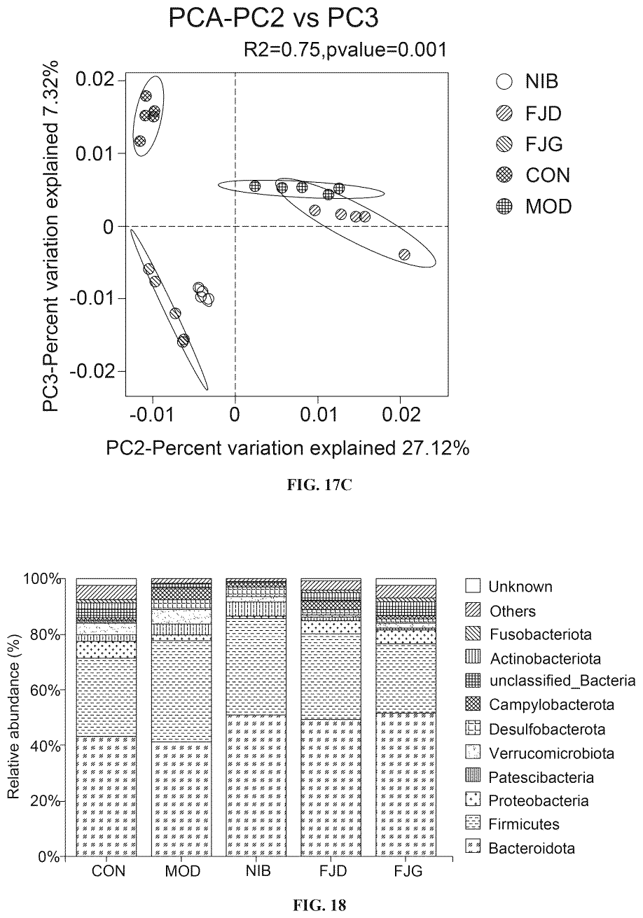

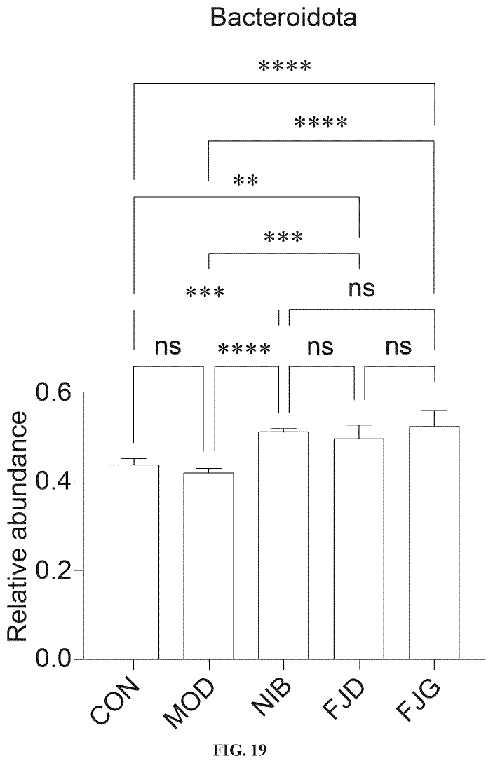

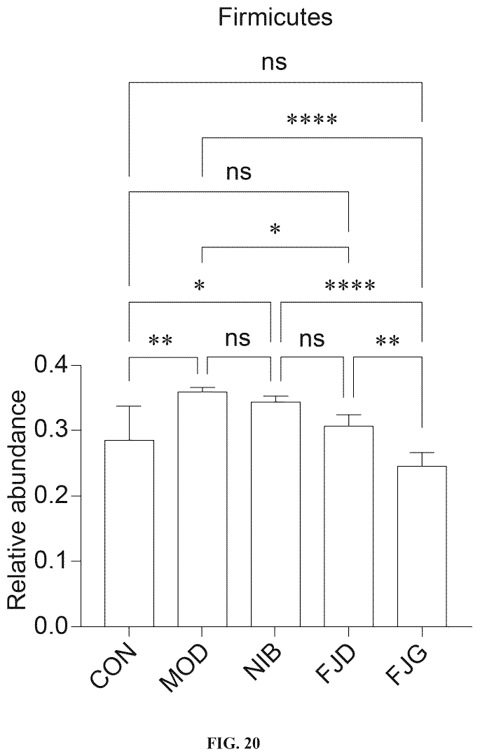

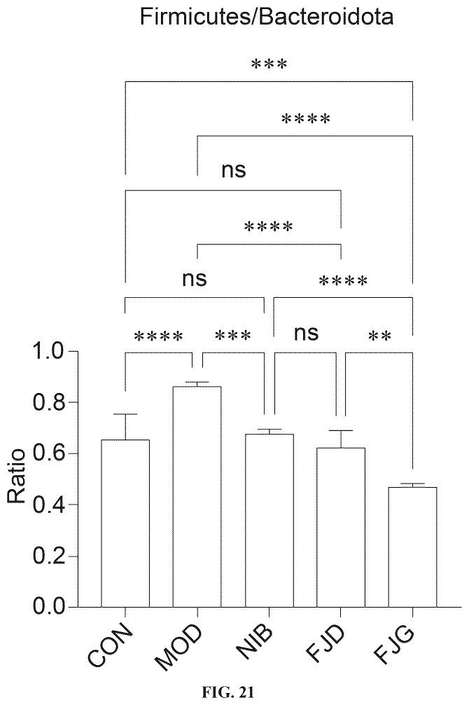

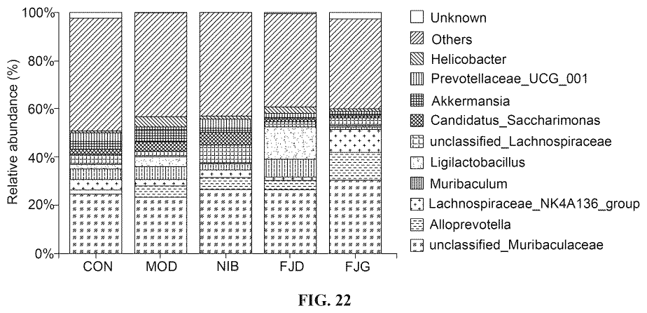

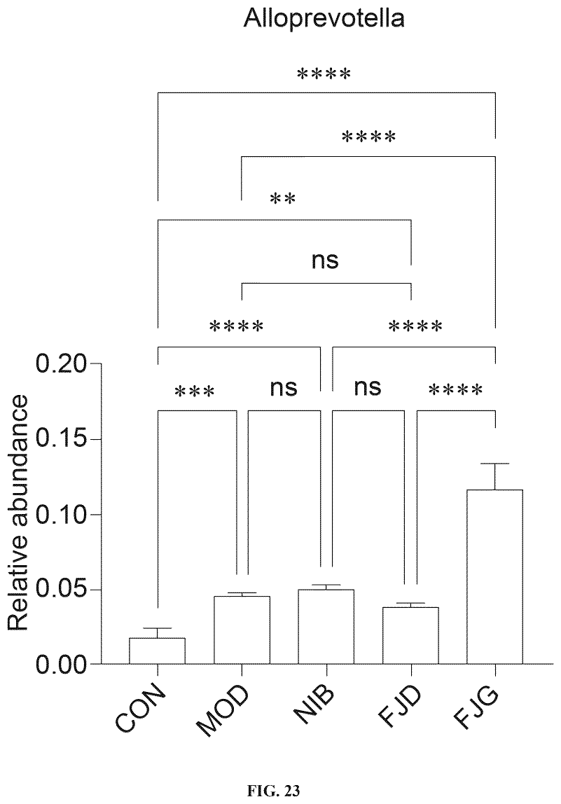

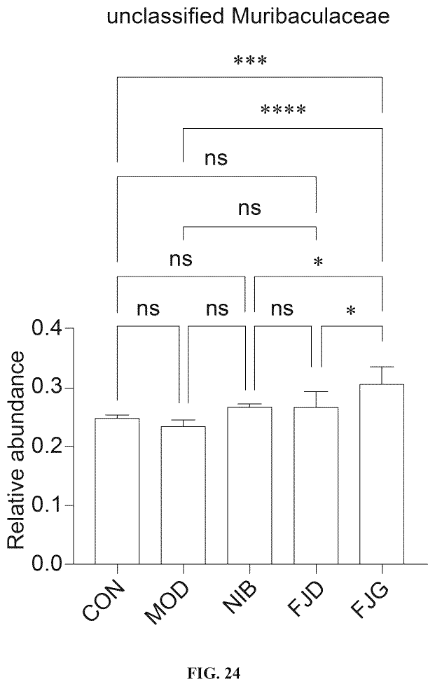

On day 27 of the consecutive drug administration, fecal samples were collected from the mice in each group. The samples were then subjected to sequencing. The sequencing results and analysis results are shown in FIGS. 14-24. All fecal samples were qualified. Five samples were selected from each group for sequencing.

It can be seen from the results in FIGS. 14-24 that the traditional Chinese medicine decoction had a given regulatory effect on the intestinal flora. The differences in the microflora between samples from different groups were analyzed at the phylum level. At the phylum level, the relative abundances of Firmicutes, Bacteroidota, Proteobacteria, and Actinobacteriota underwent significant changes. The traditional Chinese medicine decoction of the present disclosure can decrease the abundance of Firmicutes, increase the abundance of Bacteroidota, decrease the Firmicutes/Bacteroidota ratio, and increase the proportions of Proteobacteria and Actinobacteriota. The differences in the microflora between samples from different groups were analyzed at the genus level. At the genus level, the traditional Chinese medicine decoction of the present disclosure can increase the proportions of short-chain fatty acid-producing bacteria, including Alloprevotella and unclassified_Muribaculaceae.

The traditional Chinese medicine decoction has a protective effect on the lungs of bleomycin-induced pulmonary fibrosis mice, can improve the lung coefficient and lung function, ameliorate pathological changes in lung tissue, and inhibit systemic inflammation, and exerts a given regulatory effect on the intestinal flora.

The descriptions above are merely the preferred embodiments of the present disclosure. It should be noted that several improvements and modifications may also be made by those of ordinary skill in the art without departing from the principle of the present disclosure, and these improvements and modifications shall also be considered within the scope of protection of the present disclosure.

What is claimed is:

1. A traditional Chinese medicine composition for preventing and/or treating interstitial lung disease, comprising the following raw materials in parts by weight:
25-35 parts of Astragali Radix, 15-25 parts of Codonopsis Radix, 10-15 parts of Atractylodis Macrocephalae Rhizoma, 10-20 parts of Poria, 10-15 parts of Armeniacae Semen Amarum, 10-15 parts of Platycodonis Radix, 10-20 parts of Salviae Miltiorrhizae Radix et Rhizoma, 10-20 parts of Chuanxiong Rhizoma, 10-15 parts of Angelicae Sinensis Radix, 4-10 parts of Schisandrae Chinensis Fructus, and 5-12 parts of Glycyrrhizae Radix et Rhizoma.

2. The traditional Chinese medicine composition of claim 1, comprising the following raw materials in parts by weight:
28-32 parts of Astragali Radix, 16-23 parts of Codonopsis Radix, 11-14 parts of Atractylodis Macrocephalae Rhizoma, 12-17 parts of Poria, 11-13 parts of Armeniacae Semen Amarum, 11-13 parts of Platycodonis Radix, 12-18 parts of Salviae Miltiorrhizae Radix et Rhizoma, 13-17 parts of Chuanxiong Rhizoma, 11-14 parts of Angelicae Sinensis Radix, 5-7 parts of Schisandrae Chinensis Fructus, and 8-10 parts of Glycyrrhizae Radix et Rhizoma.

3. A method for preparing the traditional Chinese medicine composition of claim 1, comprising the steps of:
mixing the Astragali Radix, the Codonopsis Radix, the Atractylodis Macrocephalae Rhizoma, the Poria, the Armeniacae Semen Amarum, the Platycodonis Radix, the Salviae Miltiorrhizae Radix et Rhizoma, the Chuanxiong Rhizoma, the Angelicae Sinensis Radix, the Schisandrae Chinensis Fructus and the Glycyrrhizae Radix et Rhizoma to obtain a raw material mixture; and
mixing the raw material mixture with water and soaking the mixture, decocting the mixture for extraction and filtering the decoction mixture to obtain a filtrate.

4. The method of claim 3, further comprising the steps of:
subjecting the filtrate to a first concentration to obtain concentrate 1, allowing the concentrate 1 to cool and settle and filtering the mixture to obtain filtrate a, then subjecting the filtrate a to a second concentration and then filtering the resulting mixture to obtain concentrate 2 as the traditional Chinese medicine composition.

5. The method of claim 3, wherein a mass-to-volume ratio of the raw material mixture to water is 1:6-11; a time for the soaking is 0.5-1.5 h; the extraction is performed 1-3 times with each extraction lasting 0.5-1.5 h.

6. The method of claim 3, wherein the first concentration or the second concentration is performed at 60-90° C. under reduced pressure of −0.04 to −0.1 MPa; the concentrate 1 has a relative density of 1.04-1.06; the concentrate 2 has a relative density of 1.02-1.04.

7. A traditional Chinese medicine decoction for preventing and/or treating interstitial lung disease, comprising the traditional Chinese medicine composition of claim 1.

8. The traditional Chinese medicine decoction of claim 7, further comprising 2-4 parts of potassium sorbate.

9. The traditional Chinese medicine decoction of claim 7, wherein the traditional Chinese medicine composition comprises the following raw materials in parts by weight:
28-32 parts of Astragali Radix, 16-23 parts of Codonopsis Radix, 11-14 parts of Atractylodis Macrocephalae Rhizoma, 12-17 parts of Poria, 11-13 parts of Armeniacae Semen Amarum, 11-13 parts of Platycodonis Radix, 12-18 parts of Salviae Miltiorrhizae Radix et Rhizoma, 13-17 parts of Chuanxiong Rhizoma, 11-14 parts of Angelicae Sinensis Radix, 5-7 parts of Schisandrae Chinensis Fructus, and 8-10 parts of Glycyrrhizae Radix et Rhizoma.

10. A method for preparing the traditional Chinese medicine decoction of claim 7, comprising the steps of:
uniformly mixing a traditional Chinese medicine composition, potassium sorbate and water, boiling the mixture and then allowing the mixture to cool down to obtain the traditional Chinese medicine decoction;
wherein the traditional Chinese medicine composition is preparing a process comprising the steps of:
mixing the Astragali Radix, the Codonopsis Radix, the Atractylodis Macrocephalae Rhizoma, the Poria, the Armeniacae Semen Amarum, the Platycodonis Radix, the Salviae Miltiorrhizae Radix et Rhizoma, the Chuanxiong Rhizoma, the Angelicae Sinensis Radix, the Schisandrae Chinensis Fructus and the Glycyrrhizae Radix et Rhizoma to obtain a raw material mixture; and
mixing the raw material mixture with water and soaking the mixture, decocting the mixture for extraction and filtering the decoction mixture to obtain a filtrate.

11. A method for preventing and/or treating interstitial lung disease, or modulating the intestinal flora, comprising administering the traditional Chinese medicine composition of claim 1 to a subject in need thereof.

12. The method of claim 11, wherein the traditional Chinese medicine composition comprises the following raw materials in parts by weight:
28-32 parts of Astragali Radix, 16-23 parts of Codonopsis Radix, 11-14 parts of Atractylodis Macrocephalae Rhizoma, 12-17 parts of Poria, 11-13 parts of Armeniacae Semen Amarum, 11-13 parts of Platycodonis Radix, 12-18 parts of Salviae Miltiorrhizae Radix et Rhizoma, 13-17 parts of Chuanxiong Rhizoma, 11-14 parts of Angelicae Sinensis Radix, 5-7 parts of Schisandrae Chinensis Fructus, and 8-10 parts of Glycyrrhizae Radix et Rhizoma.


SEQUENCE LISTING (2 sequences)

SEQ ID NO: 1 - DNA, 20 bp, synthetic construct
  source 1..20: mol_type = other DNA; note = upstream primer 338F
actcctacgggaggcagcag

SEQ ID NO: 2 - DNA, 20 bp, synthetic construct
  source 1..20: mol_type = other DNA; note = downstream primer 806R
ggactachvgggtwtctaat